(19) United States
(12) Patent Application Publication
(10) Pub. No.: US 2006/0134707 A1
(43) Pub. Date: Jun. 22, 2006

(54) Monoclonal and polyclonal antibodies to equine hemoglobin and apparatus and methods using the antibodies and/or peroxidase reactions in the identification and localization of ulcers in equines

(76) Inventors: Franklin L. Pellegrini, Streetsboro, OH (US); Scott C. Anderson, Hudson, OH (US)

Correspondence Address: REINHART BOERNER VAN DEUREN S.C.;ATTN: LINDA KASULKE, DOCKET COORDINATOR, 1000 NORTH WATER STREET, SUITE 2100, MILWAUKEE, WI, 53202, US

(21) Appl. No.: 11/291,696

(22) Filed: Dec. 1, 2005

Related U.S. Application Data
(60) Provisional application No. 60/633,167, filed on Dec. 4, 2004.

(51) Int. Cl. C07K 16/42; G01N 33/567; G01N 33/53

(52) U.S. Cl. 435/7.2; 530/387.2

(57) ABSTRACT

A diagnostic and testing apparatus and related methods for the use of the same are disclosed which derive and use antibodies to equine globin and hematin in testing apparatus, kits, and methods for detecting and localizing gastric and colonic ulcers in horses. Fecal droppings from a horse to be tested are placed in a large container together with a buffered liquid solution and mixed thoroughly, following which several drops of liquid from the container are placed into a test kit. Visual markers in the test kits signify the detection of the indicators hematin and equine globin, which are respectively indicative of the presence of gastric and/or colonic ulcers.

### CROSS-REFERENCE TO RELATED APPLICATION

This application claims priority of U.S. Provisional Patent Application No. 60/633,167, which is entitled “Antibodies to Equine Globin and Equine Hematin and Apparatus and Methods Using the Antibodies in the Identification and Localization of Ulcers in Equines,” and which was filed on Dec. 4, 2004, the entirety of which application is hereby incorporated herein by reference.

### BACKGROUND OF THE INVENTION

### Field of the Invention

The present invention relates generally to diagnostic and testing apparatus and methods, and more particularly to antibodies to equine hemoglobin and hematin and the use thereof in testing apparatus, kits, and methods for detecting and localizing gastric and colonic ulcers in horses.

Prior to discussing the testing for and the diagnosis of ulcers in horses, it is beneficial to discuss the somewhat unique digestive tract anatomy of horses which contributes to a high incidence of digestive tract ulcers in horses. In the case of humans and most other animals, gastric acid is secreted in the stomach in response to eating. In contrast, horses have developed over millennia as trickle feeders (eating slowly but more or less continuously over most of the day), and their digestive systems are geared for such a diet, with a continuous production of gastric juices and bile secretion into the foregut from the liver. Thus, the stomach of a horse may be thought of as an acid pump that produces gastric acid more or less continuously through the day, whether or not the horse is being fed.

The incidence of digestive tract ulcers in performance horses has risen most sharply, from approximately twenty percent in 1920 to approximately ninety percent or better in the last decade. In racehorses, for example, as much as ninety-seven percent of the racehorse population has been reported to have digestive tract ulcers, with the percentage of show horses having digestive tract ulcers lagging only slightly behind. Even performance horse foals have been inflicted with this condition, with approximately sixty percent of performance horse foals having digestive tract ulcers. While pleasure horses have a lower incidence of digestive tract ulcers than show horses, the increasing incidence of digestive tract ulcers in the last two decades has been significant for all segments of the horse population, including pleasure horses.

A recent scientific study of a random cross-section of horses indicated that approximately fifty-five percent of them had gastric ulcers and forty percent of them had colonic ulcers. The incidences of gastric and colonic ulcers were not identical, meaning that some horses had only gastric ulcers and other horses had only colonic ulcers. However, a large percentage of the horses that had colonic ulcers also had gastric ulcers, with less than thirty percent of the horse population as a whole not having either gastric or colonic ulcers. As mentioned above, the incidence of digestive tract ulcers for show horses and racehorses is even higher than these statistics for the general horse population.

There are a number of solutions to the problem of digestive tract ulcers in horses that have been utilized in the art. Such solutions have included the use of antacids to temporarily neutralize acid in the stomach, the use of drugs to inhibit the production of gastric acid, and extended rest and a diet of forage. More recently, a novel and highly effective dietary supplement has been developed to treat and/or prevent gastric ulcers and colonic ulcers, as disclosed in U.S. patent application Ser. No. 10/435,367, filed on May 9, 2003, entitled “Dietary Supplement and Method for the Treatment and Prevention of Digestive Tract Ulcers in Equines and Other Animals,” which patent application is assigned to the assignee of the present invention, and which patent application is hereby incorporated herein by reference in its entirety.

While such treatments are available, it has remained extremely difficult to diagnose gastric ulcers in horses with a high degree of precision, and it has not been possible to diagnose colonic ulcers in horses at all. The most commonly utilized method of diagnosing equine ulcers, namely the use of symptoms (which are frequently vague, non specific signs such as weight loss, poor appetite, lethargy, or intermittent fever) combined with the perceived results of treatment, has been found to be nearly completely unreliable. This is due to the fact that there can be many potential causes of the same symptoms, and not all horses show the same or even marked symptoms of ulceration. As such, the use of this technique is often little better than a guessing game.

The only reliable way of diagnosing gastric ulcers in horses has been through the use of a three meter video endoscope, which has the significant disadvantages of being expensive, time-consuming, and stressful (both to the horse and to the trainer/owner). The cost to purchase a three meter video endoscope is quite high, and is prohibitively expensive to owners and all but the most elite of trainers. On top of the cost of the endoscope is the fact that the procedure is both cumbersome and time-consuming. Owners, trainers, and veterinarians who do not have a three meter video endoscope must consult a clinic or a veterinarian who has an endoscope. This takes additional time and expense, is even more stressful to the horse, and is frustrating since it takes the care of the horse out of the hands of the owner, trainer, or usual veterinarian.

In addition, even if a three meter video endoscope is available and is used, the results are simply not conclusive because even the most sophisticated endoscopy will miss seventy-five feet of digestive tract—the entire hindgut. The hindgut cannot be viewed using an endoscope since endoscopes of sufficient length are not available and since the use of an endoscope would require emptying the hindgut sufficiently that it would likely kill the horse. In fact, it has only been recently that the high incidence of colonic ulcers in horses has been documented, and that was done solely through the use of post-mortem visual analysis.

It is accordingly the primary objective of the present invention that it present an equine ulcer test kit and a related method for the use of the test kit which are efficacious in the diagnosis of both gastric ulcers and colonic ulcers in horses. It is a related objective of the present invention that it provide a highly specific indication as to the presence of either gastric ulcers or colonic ulcers, or both. It is another related objective of the present invention that it be highly reliable both in its identification of the existence of ulcers in a horse as well as its identification of the type(s) of the ulcers which are present in the horse, and that it not produce excessive false positive readings.

It is another objective of the present invention that the test be both simple and quick to perform, and that it require no special skill or training in order for a user to perform the test. It is a further objective of the present invention that it be entirely self-contained, requiring no laboratory analysis or additional processing equipment so that it can be performed anywhere as a field test. It is still another objective of the present invention that it provide the results of the test quickly, in minutes rather than requiring an extended time.

The equine ulcer test kit of the present invention must also be of construction which is both durable and long lasting, and it should also not require special storage conditions for the test kits in order to ensure that they have an extended shelf life. In order to enhance the market appeal of the equine ulcer test kit of the present invention, it should also be of inexpensive construction to thereby afford it the broadest possible market. Finally, it is also an objective that all of the aforesaid advantages and objectives of the equine ulcer test kit and method of the present invention be achieved without incurring any substantial relative disadvantage.

### SUMMARY OF THE INVENTION

The disadvantages and limitations of the background art discussed above are overcome by the present invention. With this invention, an equine ulcer test kit and a method of using the test kit are provided which are able to provide a highly sensitive and specific identification of the presence of either or both of gastric ulcers and colonic ulcers in horses, with the equine ulcer test kit and method clearly distinguishing between gastric ulcers and colonic ulcers. The ulcer test of the present invention is referred to as a fecal blood test, since it identifies components of blood contained in the feces of the horse being tested.

According to the teachings of the present invention, two blood components have been identified which are respectively highly indicative of the presence of a gastric ulcer or a colonic ulcer. The inventors of the present invention have determined that the presence of intact globin which is contained in feces is most likely of colonic origin. This is due to the fact that blood from a gastric ulcer (and any other blood cranial to the duodenum, for that matter) would be degraded by acids and peptides in the stomach, producing hematin (which is a molecule composed of two heme molecules which are joined together), among other products.

Thus, the presence of intact globin in the feces is indicative of the existence of a colonic ulcer, while the presence of hematin is indicative of the existence of a gastric ulcer. It will further be appreciated by those skilled in the art that an equine ulcer test kit and method using as indicators hematin and globin will provide a good indicator of both the presence and the location of one or more ulcers in the horse. The preferred form of the equine ulcer test kit and method of the present invention is an immunoassay which is designed to detect the presence of the hematin and globin indicators, and specifically the equine ulcer test of the preferred embodiment of the present invention is an Enzyme-Linked Immunosorbent Assay (an “ELISA” test), which is a method typically employed in biochemistry to detect whether or not a particular substance is present in a sample.

ELISA tests are rapid immunochemical tests that involve an antibody or an antigen (immunologic molecules) and an enzyme (a protein that catalyzes a biochemical reaction). An ELISA test is used to detect a substance that has antigenic properties, primarily proteins (as opposed to small molecules and ions such as glucose and potassium), such as antibodies, bacterial antigens, and hormones. So-called “Rapid” ELISA tests consist of a membrane having a fluid path from one end thereof which is attached to a fluid source to the other end thereof which is attached to a fluid sink, with three discrete and separated areas along the membrane.

The first area contains a labeled antibody, which is the antibody attached to a coloring agent, such as a dye or colloidal gold. The labeled antibodies move with the flow of fluid from the first area towards the second and third areas, and ultimately the fluid sink. If the substance of interest is in the fluid, it will bind to the labeled antibodies. The second area, which is typically a line extending across the membrane, contains antibodies which are attached to the membrane.

The antibodies in the second area have an affinity for (and will attract and latch onto) the substance of interest, creating a “sandwich” with the labeled antibodies and the substance of interest being bound to the antibodies in the second area, thereby creating a colored line which is a positive reading indicating the presence of the substance of interest. The more of the substance of interest contained in the fluid, the more of the labeled antibodies which will be bound with the substance of interest to the antibodies in the second area.

The third area, which is typically also a line extending across the membrane, uses a different antibody/antigen reaction that will create a colored line if the flow and volume is sufficient, regardless of the presence of the substance of interest. This acts as a control to indicate that the test system is operating properly. The third area is on the opposite side of the second area from the first area to indicate that the fluid being tested has passed across the second area, thereby indicating that the test system has been supplied with sufficient fluid being tested for the test system to work properly.

Such a test system is generically illustrated in U.S. Pat. No. 5,602,040, to May et al., which is based on two antibodies. Particles including a first group of antibodies are attached to the surface of colored latex or gold colloid particles which have been dried onto the nitrocellulose membrane at a first end thereof, and represent the first area referenced above. A second group of antibodies are attached to a nitrocellulose membrane in the form of a line, and represent the second area referenced above.

The test is performed by absorbing a liquid sample into the nitrocellulose membrane at the first end. The particles at the first area are freed by the liquid flow and the analyte to be detected binds to the antibody on said particles. At the second area, the analyte to be detected binds also to the other antibody which is present in the line, and a visible colored line is formed to show the presence of said analyte. This type of immunochromatographic test technique is based on the flow through a membrane, and may be referred to as a “lateral flow technique.” U.S. Pat. No. 5,602,040 is hereby incorporated herein by reference, as is U.S. Pat. No. 5,712,170, to Kouvonen et al., which provides an excellent summary of the art in this area.

Rapid ELISA tests are relatively inexpensive to manufacture, easy to operate, and provide rapid analyses without the need for laboratory equipment. The antibodies in ELISA tests are typically obtained by inoculating an animal with the substance of interest, after which the animal produces antibodies to that substance. This biochemical relationship is thereby utilized as the mechanism to isolate and detect the substance of interest. ELISA tests are both sensitive and specific, and compare well with radioimmune assay (“RIA”) tests. ELISA tests have the additional advantage of not needing radioisotopes or a radiation-counting apparatus.

The preferred embodiment equine ulcer test kit of the present invention includes two test strips which are contained in either one or two plastic casings. Fecal droppings from a horse to be tested are placed in a container such as a bucket or pail or a plastic bag, a solution (which may be water or water with a buffer such as salt) is added, and the mixture is swirled, stirred, or kneaded to mix it thoroughly. An applicator such as an eye dropper is used to drop several drops of liquid from the container onto the test strips in the casings.

Over a period of time which preferably ranges from approximately five minutes to approximately thirty minutes, the two test strips will provide a visual marker representing the control indicator, and, if the indicators being tested for are present, visual markers signifying the detection of those indicators which are respectively indicative of the presence of gastric and/or colonic ulcers. The present invention uses two indicators, one of which is indicative of the presence of a gastric ulcer and the other of which is indicative of the presence of a colonic ulcer. The gastric indicator may utilize a peroxidase reaction such as guaiac or tetramethylbenzidine instead of an antibody reaction.

In the preferred embodiment, the substance of interest which, when detected, will provide an indication of the presence of a colonic ulcer is equine globin, and the substance of interest which, when detected, will provide an indication of the presence of a gastric ulcer is equine hematin or heme. The equine ulcer test kit and method of the present invention thereby diagnoses gastric and/or colonic ulcers, providing an immediate basis for treatment.

It may therefore be seen that the present invention teaches an equine ulcer test kit and a related method for the use of the test kit which are efficacious in the diagnosis of both gastric ulcers and colonic ulcers in horses. The equine ulcer test kit and method of the present invention provide a highly specific indication as to the presence of either gastric ulcers or colonic ulcers, or both. The equine ulcer test kit and method of the present invention are highly reliable both in their identification of the existence of ulcers in a horse as well as their identification of the type(s) of the ulcers which are present in the horse, and do not produce excessive false positive readings.

The equine ulcer test kit and method of the present invention are both simple and quick to perform, and they require no special skill or training in order for a user to perform the test. The equine ulcer test kit of the present invention is entirely self-contained, and requires no laboratory analysis or additional processing equipment, thereby enabling it to be performed anywhere as a field test. The equine ulcer test kit and method of the present invention provide the results of the test quickly, in minutes rather than requiring an extended time.

The equine ulcer test kit of the present invention is of a construction which is both durable and long lasting, and the test kits do not require special storage conditions and have an extended shelf life. The equine ulcer test kit of the present invention is also be of inexpensive construction to enhance its market appeal and to thereby afford it the broadest possible market. Finally, all of the aforesaid advantages and objectives of the equine ulcer test kit and method of the present invention are achieved without incurring any substantial relative disadvantage.

### DESCRIPTION OF THE DRAWINGS

These and other advantages of the present invention are best understood with reference to the drawings, in which:

FIG. 1 is a somewhat schematic drawing of a horse showing the anatomy of the horse's digestive tract;

FIG. 2 is an isometric view showing an equine ulcer test kit which is constructed and used according to the teachings of the present invention and which has a first casing containing a gastric ulcer test strip and a second casing containing a colonic ulcer test strip; and

FIG. 3 is an isometric view showing an alternate embodiment equine ulcer test kit also constructed and used according to the teachings of the present invention and which has a single casing containing both a gastric ulcer test strip and a colonic ulcer test strip.

### DETAILED DESCRIPTION OF THE PREFERRED EMBODIMENT

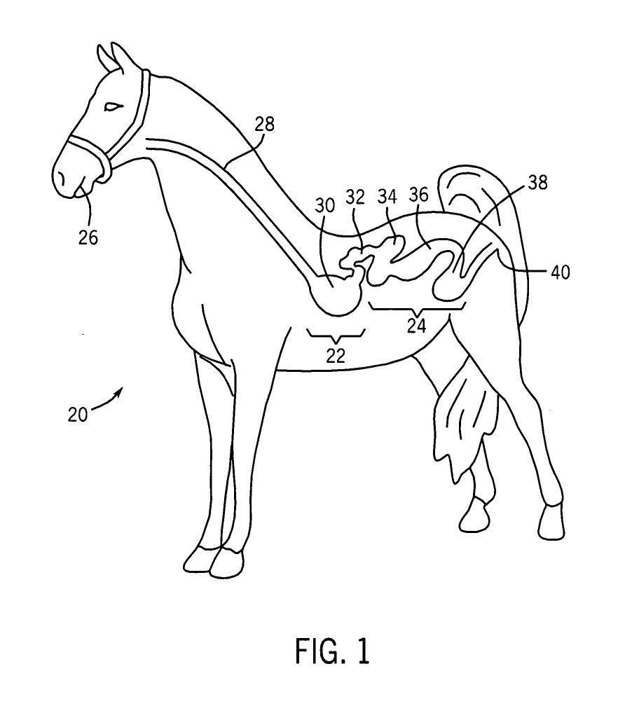

Prior to a discussion of the equine ulcer test kit and method of the present invention, it is helpful to briefly discuss the anatomy of the digestive system of a horse. Referring to FIG. 1, a side view of a horse 20 is illustrated, schematically illustrating the digestive tract of the horse. The digestive tract of the horse 20 may be separated into a foregut, which is indicated generally by the reference numeral 22, and a hindgut, which is indicated generally by the reference numeral 24.

The digestive tract of the horse 20 begins at its mouth 26, and sequentially extends through an esophagus 28 into a stomach 30 and then into a small intestine 32, which together constitute the foregut 22 of the horse 20. The foregut 22 of the horse 20 constitutes approximately thirty-five to forty percent of the relative capacity of the digestive tract of the horse 20.

From the small intestine 32, the digestive tract extends through a cecum 34, a large colon 36, and a small colon 38 which terminates in a rectum 40. These elements of the digestive tract of the horse 20 together constitute the hindgut 24 of the horse 20. The hindgut 24 constitutes approximately sixty to sixty-five percent of the relative capacity of the digestive tract of the horse 20.

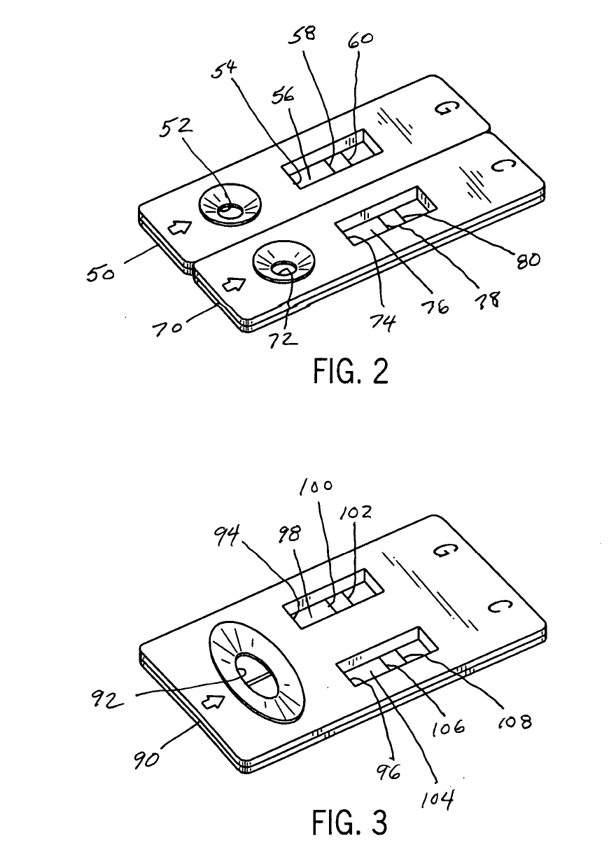

The preferred embodiment of the equine ulcer test kit of the present invention is shown in FIG. 2. A first casing 50 which contains an equine ulcer test kit for detecting gastric ulcers is illustrated. The first casing 50 has an aperture 52 located therein into which the fluid to be analyzed will be introduced. Also located in the first casing 50 is a viewing window 54 through which a test strip membrane 56 having a test indicia zone 58 and a control indicia zone 60 located therein is visible. The test indicia zone 58 will become visible when the presence of the substance of interest is detected, thereby providing an indication of the presence of a gastric ulcer.

FIG. 2 also shows a second casing 70 which contains an equine ulcer test kit for detecting colonic ulcers is illustrated. The second casing 70 has an aperture 72 located therein into which the fluid to be analyzed will be introduced. Also located in the second casing 70 is a viewing window 74 through which a test strip membrane 76 having a test indicia zone 78 and a control indicia zone 80 located therein is visible. The test indicia zone 78 will become visible when the presence of the substance of interest is detected, thereby providing an indication of the presence of a colonic ulcer. Although they are shown close together, the first casing 50 and the second casing 70 are separate in the embodiment illustrated in FIG. 2.

Referring now to FIG. 3, an alternate embodiment test kit is illustrated which has a single casing 90. The casing 90 has a single (but wider) aperture 92 located therein into which the fluid to be analyzed will be introduced. The casing 90 also has first and second viewing windows 94 and 96 located therein on opposite sides thereof. Visible through the first viewing window 94 is a test strip membrane 98 having a test indicia zone 100 and a control indicia zone 102 located therein. The test indicia zone 100 will become visible when the presence of the substance of interest is detected, thereby providing an indication of the presence of a gastric ulcer. The control indicia zone 102 will become visible when the sufficient fluid has been introduced into the casing 90 through the aperture 92.

Visible through the second viewing window 96 is a test strip membrane 104 having a test indicia zone 106 and a control indicia zone 108 located therein. The test indicia zone 106 will become visible when the presence of the substance of interest is detected, thereby providing an indication of the presence of a gastric ulcer. The control indicia zone 108 will become visible when the sufficient fluid has been introduced into the casing 90 through the aperture 92.

The construction of the test strips 56 and 76 in FIG. 2 and the test strips 98 and 104 in FIG. 3 are well known to those skilled in the art. Similarly, the construction of various other types of test kits having different casing designs are also well known to those skilled in the art. The key portions of the testing devices shown in FIGS. 2 and 3 are the type and derivation of the antibodies which are used to provide the tests for gastric and colonic ulcers, which will be discussed below.

Prior to that discussion, a brief description of the operation of the test kits shown in FIGS. 2 and 3 will be provided. A veterinarian, trainer, or horse owner collects a fecal sample (preferably en entire bowel movement) from a horse to be tested. The fecal sample is then placed into a container such as a bucket or pail or plastic bag, and an aqueous solution (which may be water or water with a buffer such as salt) is mixed therewith by swirling, stirring, or kneading. Using an eyedropper or any other convenient mechanism, the veterinarian, trainer, or horse owner then places a few drops of the fluid into the test device(s) through the apertures 52 and 72 in the casings 50 and 70, respectively for the embodiment of FIG. 1, or through the aperture 92 in the casing 90 in the embodiment of FIG. 2.

In a few minutes, the control indicia zones 60 and 80 will become visible for the embodiment of FIG. 1, or the control indicia zones 102 and 108 will become visible for the embodiment of FIG. 2, indicating the proper operation of the test kit. If globin is detected, the test indicia zone 58 will become visible for the embodiment of FIG. 1, or the test indicia zone 100 will become visible for the embodiment of FIG. 2. Similarly, if hematin is detected, the test indicia zone 78 will become visible for the embodiment of FIG. 1, or the test indicia zone 106 will become visible for the embodiment of FIG. 2. The test thereby diagnoses gastric or colonic ulcers, providing the basis for immediate treatment if either or both are detected.

While the embodiments illustrated in FIGS. 2 and 3 show separate test strip membranes 56 and 76 for the embodiment illustrated in FIG. 2, and separate test strips 98 and 104 for the embodiment illustrated in FIG. 3, those skilled in the art will immediately appreciate that it is possible to combine both tests on a single membrane.

In the preferred embodiment of the equine ulcer test kit and method of the present invention, the substance of interest which, when detected, will provide an indication of the presence of a colonic ulcer is equine globin, and the substance of interest which, when detected, will provide an indication, of the presence of a gastric ulcer is equine hematin or heme. The choice is equine globin to indicate the presence of a colonic ulcer provides specificity because globin detected in a horse's feces can only have originated from the colon. This is so because blood from gastric ulcers (and for that matter any blood cranial to the duodenum) would be degraded by acids and peptides in the stomach, thereby making it extremely unlikely that globin detected in feces would have originated from a gastric ulcer.

However, the action of acids and peptides in the stomach produce substances which include hematin and heme, among other products. Hematin or heme detected in the feces would almost certainly have originated in the stomach, and not in the colon (or for that matter from any blood caudal from the stomach), thereby making it overwhelmingly unlikely that hematin detected in feces would have originated from a colonic ulcer. Thus, those skilled in the art will appreciate that the equine ulcer test kit and method of the present invention thereby provides a mechanism and method for diagnosing gastric and/or colonic ulcers, enabling treatment for these ulcers to be provided with confidence.

### The Globin Test

The globin test is a highly sensitive monoclonal/polyclonal immunassay. There are four distinct steps in the creation of such an immunoassay, namely immunization, fusion, cloning, and production. The first stage is immunization, in which a rabbit, mouse, rat, guinea pig, or other suitable test animal is injected with equine globin derived from horse blood. This will provoke an immune reaction in the test animal, which will create copious quantities of antibodies in its blood and in its spleen.

At about six weeks, blood may be drawn from the animals and tested for antibodies using an ELISA test. This involves reacting the test animal blood with the horse sera in vitro. If antibodies are present, the ELISA will change color. If an insufficient level of antibodies are present, the test animals may require one or more booster injections of equine globin. These first bleeds can be used to produce polyclonal antibodies. This stage typically takes approximately three months.

The second stage is fusion, in which the test animals are sacrificed and their spleens are macerated to liberate the cells creating the equine antibodies. These cells may then be fused with a myeloma cell line in order to immortalize them, as described in U.S. Pat. No. 5,552,295, to Stanker et al., which patent is hereby incorporated by reference herein. Once immortalized, these cells can be cultured indefinitely to provide a continuous supply of antibodies.

The hybridomas (fused cells) are then plated out into several ninety-six-well microtiter panels. These cells are challenged with another ELISA test, and those that show the proper antibody reaction may be expanded into additional microtiter wells. These cells are further tested by ELISA testing to provide a pure line of cells producing the desired antibodies, with this stage typically taking approximately five to six weeks.

The third stage is cloning, wherein the cells that tested positive in the second stage are cloned and further tested by ELISA testing. Several cycles of cloning may be required in order to develop stable clones. These clones may then be injected into mice abdomens, where they produce ascites. The monoclonal antibodies are purified from the ascites fluid and are then ready for use. It will be readily apparent to those skilled in the art that this technique will produce novel monoclonal antibodies targeted specifically to equine globin. The third stage typically takes approximately three months.

Alternatively, chickens may be immunized with the antigen, and will produce antibody in the yolks of their eggs. This technique does not need the cloning stages mentioned above, since the chicken will lay sufficient eggs to provide the antibodies. However, the purification stages of these antibodies are similar to that described above.

The fourth and final stage is the production of test kits. The antibodies are painted onto a porous nitrocellulose or nylon membrane as is conventional in the art. When equine globin is placed on the test device it is wicked through the membrane, picks up labeled antibodies carrying a coloring agent, and is ultimately trapped by the antibodies in the test indicia zone. There, the concentration of labeled antibodies will cause a color change clearly indicating the presence of equine globin in the feces of the horse being tested. It will thus be appreciated by those skilled in the art that this novel antibody test is both extremely sensitive (the sensitivity can be as high as approximately one part in one million (one microgram per milliliter)) and specific to equine globin.

### Hematin Test

Hematin is a molecule composed of two heme molecules which are joined together. Since heme is highly conserved (being essentially the same in all mammals), it is unlikely that a test animal will develop antibodies to it. (If antibodies were created, they would attack the animal's own blood and kill it.) For this reason, in the preferred embodiment, chicken eggs are used to develop the antibody to hematin. There are four steps to this procedure, namely inoculation, collection, purification, and production

The first stage is inoculation, wherein a chicken is injected intramuscularly with equine hematin prior to egg laying. Chickens which have been injected in this manner will regularly produce eggs with the desired antibodies.

The second stage is collection, in which the eggs laid by the inoculated hens are collected, labeled, and refrigerated. Each egg may contain up to one hundred fifty milligrams of antibody in the yolk.

The third stage is purification, in which the eggs are broken and the yolk is purified with affinity-based chromatography. This elution is then further purified by ELISA testing analysis.

The fourth stage is production, wherein once the antibodies have been purified, they may be painted onto a porous nitrocellulose or nylon membrane as is conventional in the art. When equine hematin is placed on the test device it is wicked through the membrane, picks up labeled antibodies carrying a coloring agent, and is ultimately trapped by the antibodies in the test indicia zone. There, the concentration of labeled antibodies will cause a color change clearly indicating the presence of hematin in the feces of the horse being tested. It will be appreciated by those skilled in the art that this antibody test is extremely sensitive (the sensitivity can be as high as approximately one part in one million (one microgram per milliliter)) and specific to hematin.

Alternatively, a highly sensitive guaiac or tetramethylbenzidase (TMB) reaction may be used to detect heme or hematin. In this embodiment, the liquid would be wicked past the quaiac or TMB and a peroxide indicator would be applied to the test window which would change color in the presence of heme or hematin.

### The Combination Test

By combining the hematin test with the globin test in a single test kit, a simple, inexpensive, highly sensitive, and diagnosis-specific test for equine ulcers may be created. Alternatively, the two tests may be provided in separate test kits. In the preferred embodiment, the two test strips are connected to a single well, so that a single application of the fecal liquid will suffice for both.

If the hematin test is positive, a gastric ulcer is indicated. If the globin test is positive, a colonic ulcer is indicated. If both results are positive, then simultaneous gastric and colonic ulcers are indicated. It will be appreciated by those skilled in the art that this dual test can provide a convenient, non-invasive test for gastric ulcers (which are currently difficult and expensive to diagnose) as well as colonic ulcers (for which no current test capable of providing an accurate diagnosis exists).

It may therefore be appreciated from the above detailed description of the preferred embodiment of the present invention that it teaches an equine ulcer test kit and a related method for the use of the test kit which are efficacious in the diagnosis of both gastric ulcers and colonic ulcers in horses. The equine ulcer test kit and method of the present invention provide a highly specific indication as to the presence of either gastric ulcers or colonic ulcers, or both. The equine ulcer test kit and method of the present invention are highly reliable both in their identification of the existence of ulcers in a horse as well as their identification of the type(s) of the ulcers which are present in the horse, and do not produce excessive false positive readings.

The equine ulcer test kit and method of the present invention are both simple and quick to perform, and they require no special skill or training in order for a user to perform the test. The equine ulcer test kit of the present invention is entirely self-contained, and requires no laboratory analysis or additional processing equipment, thereby enabling it to be performed anywhere as a field test. The equine ulcer test kit and method of the present invention provide the results of the test quickly, in minutes rather than requiring an extended time.

The equine ulcer test kit of the present invention is of a construction which is both durable and long lasting, and the test kits do not require special storage conditions and have an extended shelf life. The equine ulcer test kit of the present invention is also be of inexpensive construction to enhance its market appeal and to thereby afford it the broadest possible market. Finally, all of the aforesaid advantages and objectives of the equine ulcer test kit and method of the present invention are achieved without incurring any substantial relative disadvantage.

Although the foregoing description of the present invention has been shown and described with reference to particular embodiments and applications thereof, it has been presented for purposes of illustration and description and is not intended to be exhaustive or to limit the invention to the particular embodiments and applications disclosed. It will be apparent to those having ordinary skill in the art that a number of changes, modifications, variations, or alterations to the invention as described herein may be made, none of which depart from the spirit or scope of the present invention. The particular embodiments and applications were chosen and described to provide the best illustration of the principles of the invention and its practical application to thereby enable one of ordinary skill in the art to utilize the invention in various embodiments and with various modifications as are suited to the particular use contemplated. All such changes, modifications, variations, and alterations should therefore be seen as being within the scope of the present invention as determined by the appended claims when interpreted in accordance with the breadth to which they are fairly, legally, and equitably entitled.

What is claimed is:

1. An anti-equine globin antibody which specifically binds to equine globin.

2. An anti-equine globin antibody as defined in claim 1, wherein said anti-equine globin antibody binds equine globin with a sensitivity equal to or better than approximately one part in one million (one microgram per milliliter).

3. An anti-equine globin antibody as defined in claim 1, wherein the anti-equine globin antibody is a monoclonal antibody.

4. An anti-equine globin antibody as defined in claim 1, wherein the anti-equine globin antibody is a polyclonal antibody.

5. An anti-equine globin antibody as defined in claim 1, wherein the anti-equine globin antibody is isolated from the macerated spleens of inoculated rabbits, mice, rats, or guinea pigs.

6. An anti-equine hematin antibody which specifically binds to equine hematin.

7. An anti-equine hematin antibody as defined in claim 6, wherein said anti-equine hematin antibody binds equine hematin with a sensitivity equal to or better than approximately one part in one million (one microgram per milliliter).

8. An anti-equine hematin antibody as defined in claim 6, wherein the anti-equine hematin antibody is a monoclonal antibody.

9. An anti-equine hematin antibody as defined in claim 6, wherein the anti-equine hematin antibody is a polyclonal antibody.

10. An anti-equine hematin antibody as defined in claim 6, wherein the anti-equine hematin antibody is isolated from inoculated chicken eggs.

11. A hybridoma cell line which produces and secretes monoclonal antibodies which specifically bind to equine globin.

12. A hybridoma cell line which produces and secretes monoclonal antibodies which specifically bind to equine hematin.

13. A method of producing an antibody that specifically binds to equine globin.

14. A method of making an anti-equine globin antibody as defined in claim 13, comprising expressing globin antibodies from immune cells which have been hybridized with immortal myeloma lines.

15. A method of making an anti-equine globin antibody as defined in claim 14, wherein said immune cells are isolated from the macerated spleens of inoculated rabbits, mice, rats, or guinea pigs.

16. A method of producing an antibody that specifically binds to equine hematin.

17. A method of making an anti-equine hematin antibody as defined in claim 16, comprising expressing hematin antibodies from immune cells which have been hybridized with immortal myeloma lines.

18. A method of making an anti-equine hematin antibody as defined in claim 16, wherein said immune cells are isolated from inoculated chicken eggs.

19. A rapid test kit for the detection and localization of ulcers in equines, comprising:
a first test strip for a rapid immunoassay or peroxidase reaction to detect the presence of a gastric ulcer in an equine; and
a second test strip for a rapid immunoassay to detect the presence of a colonic ulcer in an equine.

20. A rapid test kit as defined in claim 19, wherein said first test strip detects the presence of hematin which is indicative of the presence of a gastric ulcer in an equine.

21. A rapid test kit as defined in claim 19, wherein said first test strip also contains a control indicator to demonstrate that the test has been properly performed.

22. A rapid test kit as defined in claim 19, wherein said second test strip detects the presence of equine globin which is indicative of the presence of a gastric ulcer in an equine.

23. A rapid test kit as defined in claim 22, wherein said second test strip also contains a control indicator to demonstrate that the test has been properly performed.

24. A rapid test kit for the detection and localization of ulcers in equines, comprising:
a first test strip for a rapid immunoassay or peroxidase reaction to detect the presence of hematin which is indicative of the presence of a gastric ulcer in an equine; and
a second test strip for a rapid immunoassay to detect the presence of equine globin which is indicative of the presence of a colonic ulcer in an equine.

25. A rapid test kit as defined in claim 24, wherein said first test strip also contains a control indicator to demonstrate that the test has been properly performed, and wherein said second test strip also contains a control indicator to demonstrate that the test has been properly performed.

26. A rapid test kit as defined in claim 24, wherein said first and second test strips comprise:
a single test strip for detecting both the presence of hematin and the presence of equine globin.
